(19) United States
(12) Patent Application Publication
(10) Pub. No.: US 2012/0048282 A1
(43) Pub. Date: Mar. 1, 2012

(54) Sleep apnea headstrap apparatus

(76) Inventor: Thomas A. Maddox, Woodbine, NJ (US)

(21) Appl. No.: 12/806,921

(22) Filed: Aug. 24, 2010

(51) Int. Cl. A61F 5/56

(52) U.S. Cl. 128/848

(57) ABSTRACT

An adjustable headstrap apparatus maintains the mouth of a user in a closed position to prevent sleep apnea. The apparatus utilizes two floating brackets positioned on opposite sides of the user's head. Each bracket has three internal openings for receiving a first strap member having a chin support for supporting the chin and lower head of the user, a second strap member to maintain the apparatus on the head of the user, and a third strap member to be used around the back of the head of the user. Each strap member has an adjustment component, for instance a Velcro® attachment, which allows the length of the members to be adjustable through the brackets, so that the head strap apparatus can be sized for a comfortable custom fit around the head of the user. The head strap apparatus is thus comfortably and securely positioned on the head of the user to maintain the mouth closed during sleep and rest periods.

### BACKGROUND OF THE INVENTION

Sleep apnea is a serious condition in which there is a complete cessation of breathing during periods of sleep or rest. Some 18 million Americans are believed to be afflicted with sleep apnea, which results in extreme fatigue and, often, falling asleep during the day. Sleep apnea also is responsible for cloudy thinking and a slowing of motor skills. It is believed that sleep apnea is a cause of high blood pressure and a higher incidence of heart disease and stroke. During apnea, oxygen levels in the blood may fall, causing long term problems with the heart and brain. In addition, many cases of sudden death while sleeping are a direct result of sleep apnea.

There are two main types of sleep apnea, central and obstruction, and there are conditions which exhibit symptoms of combinations of the two. Central sleep apnea is caused by a disruptive condition in the brain. Obstructive sleep apnea is caused by a closing of the airway when the soft tissues in the neck and throat relax during sleep. This is the same mechanism that causes snoring. Obstructive sleep apnea is more likely to occur in obese people and those with large necks. The obstruction causes the patient to cease breathing for periods of time. The urge to breath causes the person to arouse from sound sleep, often abruptly, awakening completely. Depending on how many apnea episodes occur during the night will dictate the depth and amount of sleep an individual gets.

While there is no current cure for sleep apnea, it has been found that losing weight can often improve the condition. The symptoms can also be treated with breathing devices called Continuous Positive Airway Pressure (CPAP) and Bi-level Positive Airway Pressure (BiPAP) machines. These devices deliver positive air flow to a patient's airway, forcing the soft tissue open to allow normal breathing exchange, resulting in deeper and non-disrupted sleep.

Some CPAP and BiPAP machines use a nasal air delivery device which requires the patient to keep his or her mouth closed. If the mouth falls open, the positive pressure delivered air escapes before forcing the airway open and the therapy is rendered ineffective. For this reason, CPAP and BiPAP users often use a headstrap/chin support to keep the mouth in a closed position during sleep therapy.

Unfortunately, the headstrap/chin support devices currently available are not effective in ensuring that the mouth remains closed during sleep or rest periods. Such devices routinely fall off the head due to improper tensioning angles or because the material which is used slides easily over hair. Cinching the straps on these devices tight enough to be held on the head usually causes extreme and discomforting pain on the chin and jaw, since the jaw is compelled into an unnatural and painful position. When too much pressure is applied by straps in order to hold the chin and mouth closed, the jaw can also be pushed either too far backwards or upwards, again resulting in great discomfort. This defeats the object of the therapy, which is primarily to promote sleep and rest. Because the straps in most existing headstrap/chin supporting devices are fixed, the user cannot adjust the devices to fit his or her jaw shape and size. Since each individual's head and face is different, devices which do not adjust are seriously flawed.

Of course when the straps of existing devices move out of position or fall or slide completely off the head, the mouth falls open and the air forced in the nose of the patient by the CPAP or BiPAP device flows rapidly out of the mouth. Inflation of airway is not maintained and the apnea is not corrected. The large and rapid escape of air out of the mouth also causes a drying of the mouth and throat, which will eventually wake the person, again defeating the therapy.

Many of the currently available headstrap/chin devices are also quite cumbersome to put on and remove, especially while wearing a CPAP or BiPAP apparatus. The donning and removal processes are often difficult when such apparatus are being utilized.

### SUMMARY OF THE INVENTION

It is thus the object of the present invention to overcome the disadvantages and limitations of existing sleep apnea preventing headstrap/chin devices.

It is the object of the present invention to provide a sleep apnea headstrap apparatus which is most effective in maintaining the mouth of the user closed during sleep or rest periods.

It is another object of the present invention to provide a sleep apnea headstrap apparatus which can be comfortably and securely positioned on the head of a user to prevent sleep apnea, whether utilized with or without a breathing assist device.

It is still another object of the present invention to provide a sleep apnea headstrap apparatus which is fully and readily adjustable to comfortably custom fit the size and shape of the head and face of the user.

It is a further object of the present invention to provide a sleep apnea headstrap apparatus which can be easily put on, adjusted, and removed by the user.

These and other objects are accomplished by the present invention, an adjustable headstrap apparatus for maintaining the mouth of a user in a closed position to prevent sleep apnea. The apparatus utilizes two floating brackets positioned on opposite sides of the user's head. Each bracket has three internal openings for receiving a first strap member having a chin support for supporting the chin and lower head of the user, a second strap member to maintain the apparatus on the head of the user, and a third strap member to be used around the back of the head of the user. Each strap member has an adjustment component, for instance a Velcro® attachment, which allows the length of the members to be adjustable through the brackets, so that the head strap apparatus can be sized for a comfortable custom fit around the head of the user. The head strap apparatus is thus comfortably and securely positioned on the head of the user to maintain the mouth closed during sleep and rest periods.

The novel features which are considered as characteristic of the invention are set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 is a perspective view of the sleep apnea headstrap apparatus of the present invention.

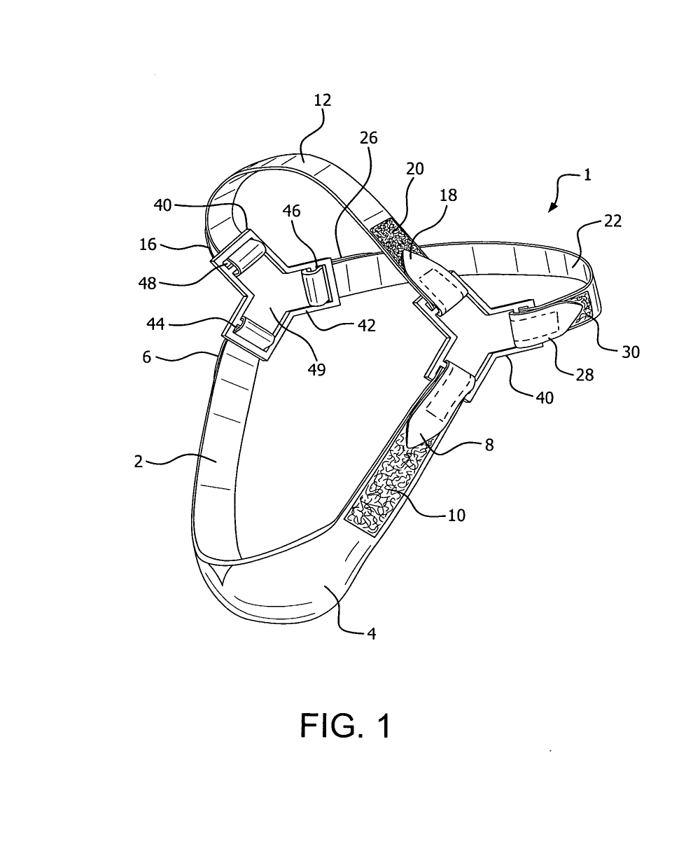

FIG. 2 shows the sleep apnea headstrap apparatus of the present invention in place on the head of a user.

FIG. 3 shows an alternate sleep apnea headstrap of the present invention in place on the head of a user.

### DETAILED DESCRIPTION OF THE INVENTION

Headstrap apparatus 1 of the present invention comprises jaw supporting strap member 2, with chin support component 4, top head strap member 12, and rear strap member 22. Ends 6 and 8 of strap member 2 each comprise Velcro® or equivalent attachments 10, which allow the length of the strap member to be readily adjustable, as discussed in further detail hereinafter. In like fashion, ends 16 and 18 of strap member 12, and ends 26 and 28 of strap member 22, each comprise length adjustment attachments 20 and 30 respectively.

Bracket 40, a separate component of headstrap apparatus 1, is a unitary, integrally formed element of rigid, lightweight metal, strong plastic, or similar material. Bracket 40 comprises outer frame 42, lower internal opening 44, rear internal opening 46, upper internal opening 48, and large central opening 49.

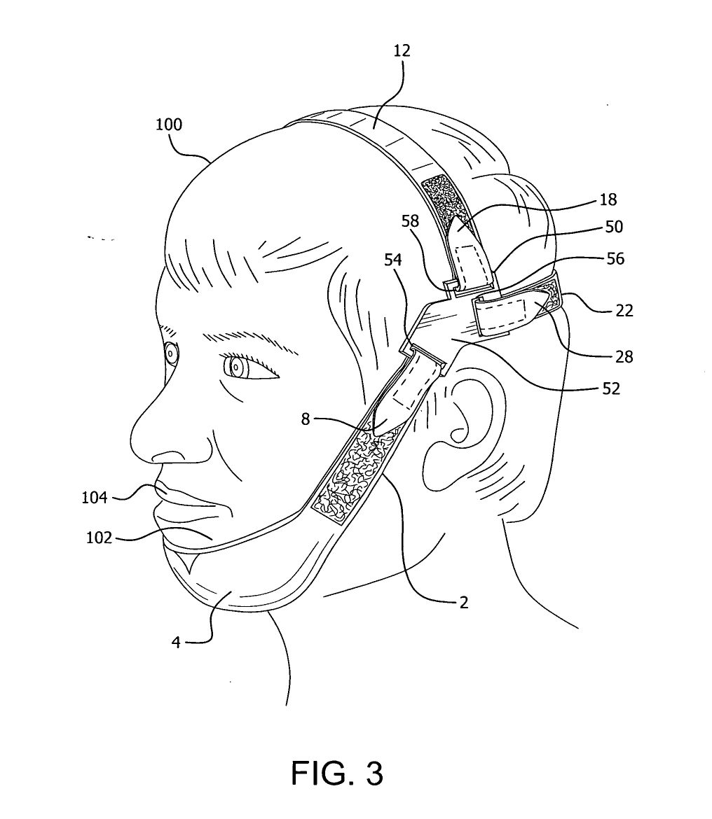

Alternate bracket 50, shown in FIG. 3, is also a unitary, integrally formed element of like material comprising main body 52 having lower internal opening 54, rear internal opening 56, and upper internal opening 58. Rear opening 56 is perpendicular to upper opening 58, and lower opening 54 is set at a non-right angle in relation to the two other openings. Headstrap apparatus 1 is designed to be used with either two brackets 40 or two brackets 50, one bracket being located on opposite sides of the head of user 100.

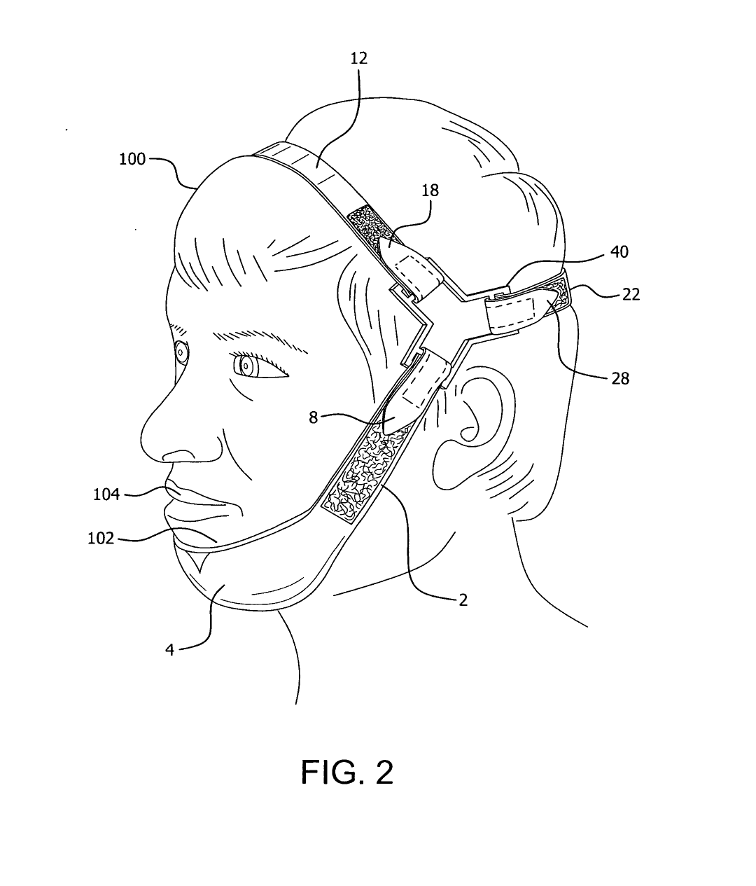

As depicted in FIGS. 2 and 3, ends 6 and 8 of strap member 2 are inserted into lower opening 44 of bracket 40 or 54 of bracket 50; ends 16 and 18 of strap member 12 are inserted into upper opening 48 of bracket 40 or 58 of bracket 50; and ends 26 and 28 of strap member 22 are inserted into rear opening 46 of bracket 40 or 56 of bracket 50.

In operation, a user positions headstrap apparatus over his or her head 100, such that chin support 4 is snugly against the chin 102 area. The lengths of strap members 2, 12 and 22 are then alternately adjusted on user's head 100, so that headstrap apparatus 1 maintains mouth 104 of the user in a closed position, while ensuring that the headstrap apparatus itself remains comfortably, but securely on the user's head 100. This comfortable fit is accomplished by appropriate adjustment of strap members 2, 12, and 22; that is by varying the lengths of the strap members and securing them in position with brackets 40 or 50, via attachments 10, 20, and 30, respectively. In this respect, brackets 40 and 50, are moveable to a plurality of positions by adjustment of the lengths of the strap members, and so are considered to be floating brackets. That is, they are literally suspended by and moveably “float” along user's head 100, as strap member adjustments are made. Once in position, headstrap apparatus 1 remains fixed on user's head 100 to maintain mouth 104 closed.

The design of bracket 50 is especially convenient for readily positioning headstrap apparatus 1. The locations of openings 54, 56, and 58 make the placement and adjustment of strap members 2, 12, and 22 through bracket 50 relatively easy, so that headstrap apparatus 1 can be fitted securely and comfortably on the user's head 102.

While two floating bracket designs are disclosed herein, the headstrap apparatus of the present invention should not be considered so restricted. Equivalent bracket designs which ensure comfortable fit of the invention are contemplated as well.

Headstrap apparatus 1, when positioned properly on the user, will successfully maintain the mouth of the user closed during sleeping, and thus greatly assist in preventing sleep apnea. The three strap member/floating bracket system allows the user to readily put on the apparatus and adjust the angle at which the straps are positioned on the head, thus permitting customized adjustment and support for all head/chin shapes and sizes. This system also allows the apparatus to be positioned on the user's head so as to avoid head and facial areas which could cause discomfort, e.g. ears, cheek bones, temples, pressure points, etc., and to avoid putting pressure on CPAP or BiPAP devices which may be used, thereby ensuring for an effective fit of the breathing device. It is also contemplated that the strap members of the headstrap apparatus can be used with removeable, cushioned pads, to provide additional comfort padding. This will also assist in securing the apparatus in place over long hair and prevent hair from becoming pulled and tangled.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modifications and changes may be made without departing from the spirit of the invention.

1. A headstrap apparatus for maintaining the mouth of a user in a closed position to prevent sleep apnea comprising:
two brackets, each comprising three internal openings therethrough;
first strap means extending through one of the openings of each bracket for supporting the chin of the user, said strap means having first means to adjust the length of the strap means;
second strap means extending through a second of the openings of each bracket for maintaining the headstrap apparatus on the head of the user, said second strap means having second means to adjust the length of the second strap means; and
third strap means extending through the third opening of each bracket for maintaining the headstrap apparatus on the head of the user, said third strap means having third means to adjust the length of the third strap means, each bracket being configured to be moveable to a plurality of positions along the sides of the head of the user, the location of each said position being dependent on the lengths of the first, second, and third strap means, whereby the length of the first, second, and third strap means are each individually adjustable through the brackets to secure the headstrap apparatus on the head of the user and, at all times, to maintain the mouth of the user in a closed position.

2. The headstrap apparatus as in claim 1 wherein the second strap means is configured to extend around the back of the head of the user.

3. The headstrap apparatus as in claim 1 wherein the third strap means is configured to extend over the top of the head of the user.

4. A headstrap apparatus as in claim 3 wherein the second strap means is configured to extend around the back of the head of the user.

5. The headstrap apparatus as in claim 1 wherein the first strap means comprises a chin support.

6. The headstrap apparatus as in claim 1 wherein the each bracket comprises a lower internal opening, an upper internal opening, and a lateral internal opening.

7. The headstrap apparatus as in claim 6 wherein the lateral internal opening is substantially perpendicular to the top internal opening.

8. The headstrap apparatus as in claim 1 wherein one bracket is configured to be positioned on each side of the head of the user.
